(19) United States
(12) Patent Application Publication
(10) Pub. No.: US 2016/0380204 A1
(43) Pub. Date: Dec. 29, 2016

(54) HOLE TRANSPORT LAYER COMPOSITION FOR SOLAR CELL, PREPARATION METHOD THEREOF AND SOLAR CELL COMPRISING THE SAME

(71) Applicants: KOREA INSTITUTE OF MACHINERY AND MATERIALS, Daejeon (KR); PUSAN NATIONAL UNIVERSITY INDUSTRY-UNIVERSITY COOPERATION FOUNDATION, Busan (KR)

(72) Inventors: Myungkwan SONG, Ulsan (KR); Chang Su KIM, Gyeongsangnam-do (KR); Dong Ho KIM, Gyeongsangnam-do (KR); Sung-Ho JIN, Busan (KR)

(21) Appl. No.: 15/175,641

(22) Filed: Jun. 7, 2016

(30) Foreign Application Priority Data
Jun. 29, 2015 (KR) 1020150091901

(51) Int. Cl. H01L 51/00; H01L 51/42; C07D 209/86; H01L 51/44

(52) U.S. Cl. CPC H01L 51/0061; H01L 51/441; H01L 51/4233; C07D 209/86; H01L 51/006; H01L 51/0072; H01L 51/0056; H01L 51/0058

(57) ABSTRACT

A hole transport layer composition is for a solar cell, a preparation method is thereof, and there is a solar cell comprising the same. More precisely, a hole transport layer composition for solar cell comprises the compound represented by formula 1. The hole transport layer composition can be used as a material for hole transport layer for solar cell which displays the improved power conversion efficiency than the conventional material. In addition, the hole transport layer composition demonstrates a high hole mobility, a proper energy level, a thermo-stability, and an excellent solubility, so that it can provide a similar or higher power conversion efficiency than the conventional spiro-OMeTAD. A solar cell comprising the hole transport layer composition displays a higher power conversion efficiency because the hole transport layer composition for solar cell includes a low-molecular material having a high charge carrier mobility instead of including a high-molecular material.

### BACKGROUND OF THE INVENTION

1. Field of the Invention

The present invention relates to a hole transport layer composition for solar cell, a preparation method thereof, and the solar cell comprising the same.

2. Description of the Related Art

It has never been requested as much as it is requested now to develop a solar energy that can be provided continuously, considering the worries of exhaustion of fossil fuel, global warming and weather changes due to the abuse of fossil fuel, and safety concern about nuclear energy. The solar energy that the sun can deliver to the earth is total 10⁵ TW per unit hour at average. Only a part of it is sufficient for the total usage on earth, which is assumed to be 20 TW, in 2020. Not all the energy from the sun is usable of course, but it is still a most fascinating new regenerable energy due to the minor area proponderance and pro-environmental advantage.

Solar cell technology is the technique that is to change the light directly into electric energy. Most of the commercialized solar cell is the inorganic solar cell using an inorganic matter such as silicon. However, such an inorganic solar cell has a disadvantage of increasing the production costs due to the complicated production process and the high price of the raw material. Therefore, studies have been actively going on to develop an organic solar cell that has advantage of low production costs resulted from the simplified production process and using a low priced material.

Perovskite solar cell is on the spotlight because of its excellent photoelectric cell characteristics, low costs, and comparatively simple process. The Perovskite solar cell that does not include a hole transporting material displays a lower charge extraction and charge recombination on the interface than the Perovskite solar cell comprising a hole transport material. To increase the power conversion efficiency (PCE), it is necessary to increase charge extraction and to alleviate unwanted charge recombination on the interface. To do so, the role of the hole transporting material (HTM) is very important in Perovskite solar cell.

Studies have been actively going on to increase the power conversion efficiency, one of the major characteristics of Perovskite solar cell. Recently, it was succeeded to increase the power conversion efficiency of Perovskite solar cell to 15% by using spiro-OMeTAD as a hole transporting material. However, the spiro-MOeTAD synthesis is complicated and requires a high price but displays a low charge carrier mobility, resulting in the limitation of generalization of the solar cell. A polymer-based hole transporting material is widely used. However, it demonstrates s series of problems such as the possible damage in device safety due to the acidic environment, the difficulty in regulation of the molecular weight of the polymer, polydispersity, and stereoregularity that can directly affect the performance of a solar cell, the complexity of the synthesis or purification process, and the low charge carrier mobility.

Therefore, it is necessary to design and develop a small molecule hole transporting material that can be an efficient alternative in order to achieve high efficiency.

In the course of study to increase efficiency and stability of the Perovskite solar cell and organic solar cell by using a novel hole transporting material, the present inventors succeeded in designing and synthesis of a novel low-molecular hole transporting material having a chemical structure wherein phenylcarbazole and fluorene group form a main bone using a nitrogen atom as a connector and spirobifluorene derivative is located as an end capper, leading to the completion of this invention.

### SUMMARY OF THE INVENTION

It is an object of the present invention to provide a hole transport layer composition for solar cell, a preparation method thereof, and a solar cell comprising the same.

To achieve the above object, the present invention provides a hole transport layer composition comprising the compound represented by formula 1.

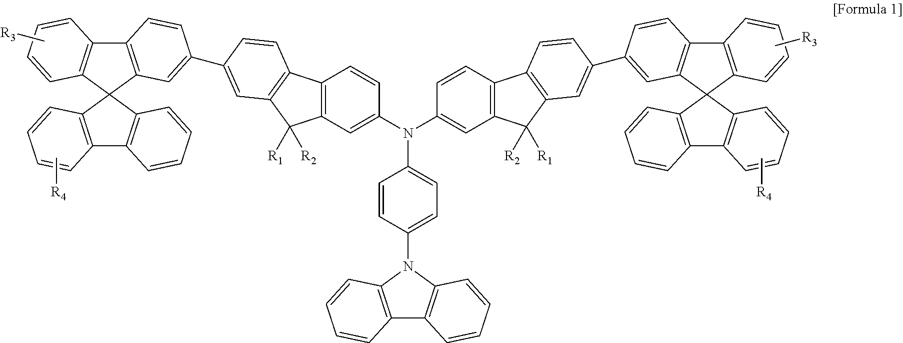

(In the formula 1,

R₁ and R₂ are independently hydrogen or C₁˜C₂₀ alkyl group;

R₃ is hydrogen, C₁˜C₁₀ alkyl or cyano group; and

R₄ is hydrogen or C₁˜C₁₀ alkyl group.)

The present invention also provides a method for preparing a hole transport layer composition for solar cell comprising the following steps as shown in reaction formula 1:

preparing the compound represented by formula 3 by reacting carbazole represented by formula 2 with 4-iodoaniline (step 1);

preparing the compound represented by formula 5 by reacting the compound of formula 3 prepared in step 1) with the compound represented by formula 4 (step 2);

preparing the compound represented by formula 7 by reacting the compound of formula 5 prepared in step 2) with the compound represented by formula 6 (step 3); and

preparing the compound represented by formula 9 by reacting the compound of formula 7 prepared in step 3) with the compound represented by formula 8 (step 4);

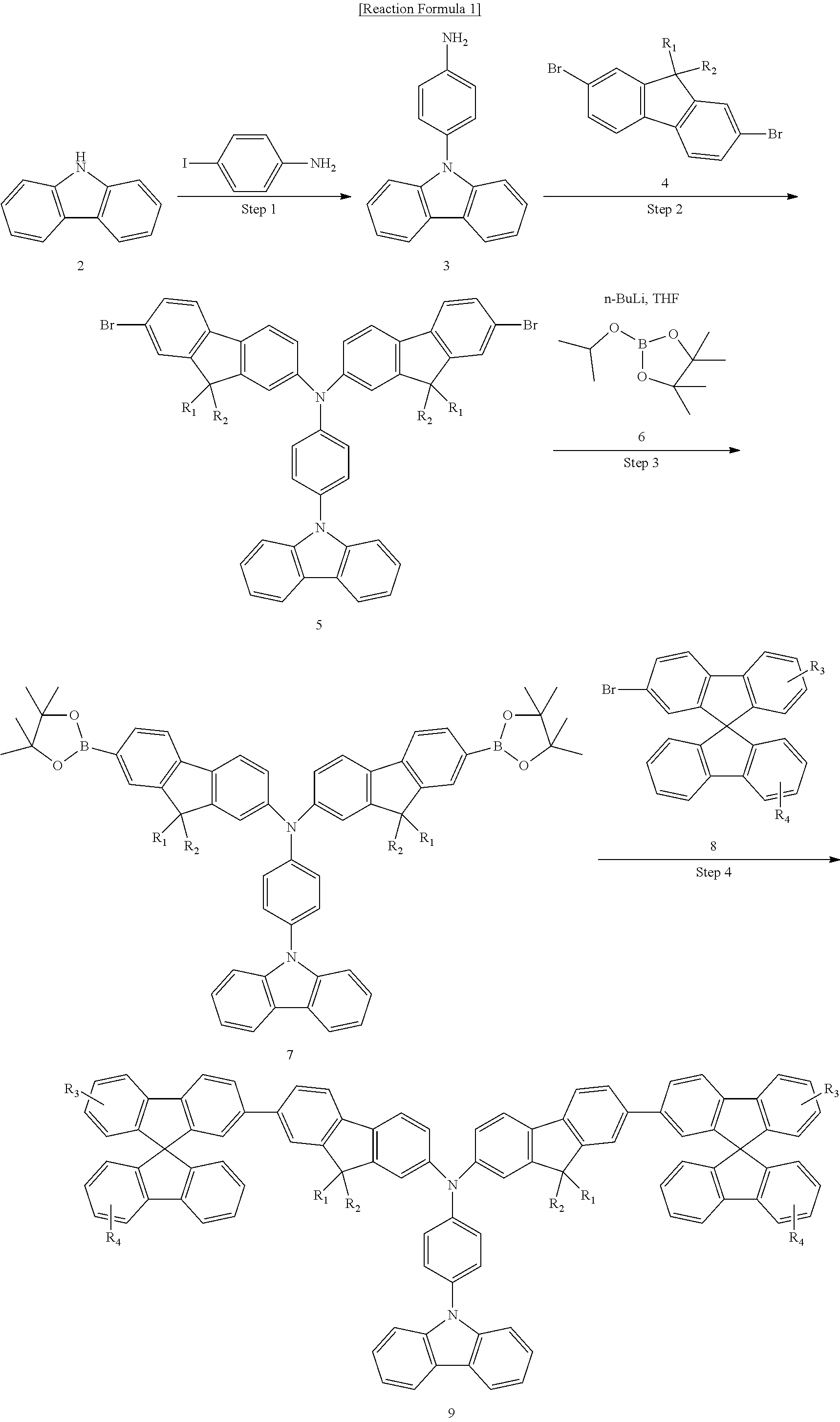

(In the reaction formula 1,

R₁ and R₂ are independently hydrogen or C₁˜C₂₀ alkyl group;

R₃ is hydrogen, C₁˜C₁₀ alkyl or cyano group; and

R₄ is hydrogen or C₁˜C₁₀ alkyl group.)

In addition, the present invention provides a solar cell comprising the compound represented by formula 1 as a hole transport layer.

### Advantageous Effect

The power conversion efficiency of a solar cell can be improved by using the hole transport layer composition of the present invention as a hole transport layer material. In addition, the hole transport layer composition of the invention demonstrates a high hole mobility, a proper energy level, a thermo-stability, and an excellent solubility, so that it can provide a similar or higher power conversion efficiency than the conventional spiro-OMeTAD. That is, a solar cell comprising the hole transport layer composition of the invention displays a higher power conversion efficiency because the hole transport layer composition of the invention for solar cell includes a low-molecular material having a high charge carrier mobility instead of including a high-molecular material.

### BRIEF DESCRIPTION OF THE DRAWINGS

The application of the preferred embodiments of the present invention is best understood with reference to the accompanying drawings, wherein:

FIGS. 1(a)-1(d) are diagrams illustrating the structure and the energy level of a solar cell;

FIG. 2 is a graph illustrating the UV-VIS absorption spectrum of a hole transporting material of a solar cell;

FIG. 3 is a graph illustrating the UV-VIS absorption fluorescence spectrum of the hole transport layer material for solar cell in the states of chloroform solution;

FIG. 4 is a graph illustrating the UV-VIS absorption fluorescence spectrum of the hole transport layer material for solar cell in the states of film;

FIG. 5 is a graph illustrating the result of cyclic voltammetry with the hole transporting material for solar cell;

FIG. 6 is a graph illustrating the results of thermogravimetric analysis with the hole transporting material for solar cell;

FIG. 7 is a graph illustrating the results of differential scanning calorimetry with the hole transporting material for solar cell;

FIG. 8 is a graph illustrating the photocurrent density-voltage (J-V) curve of Perovskite solar cell according to a hole transporting material;

FIG. 9 is a graph illustrating the current efficiency (IPCE)-wavelength curve of Perovskite solar cell according to a hole transporting material;

FIG. 10 is a graph illustrating the photocurrent density-voltage (J-V) curve of the bulk heterojunction organic solar cell according to a hole transporting material.

FIG. 11 is a graph illustrating the current efficiency (IPCE)-wavelength curve of the bulk heterojunction organic solar cell according to a hole transporting material.

### DESCRIPTION OF THE PREFERRED EMBODIMENTS

Hereinafter, the present invention is described in detail.

The present invention provides a hole transport layer composition for solar cell comprising the compound represented by formula 1.

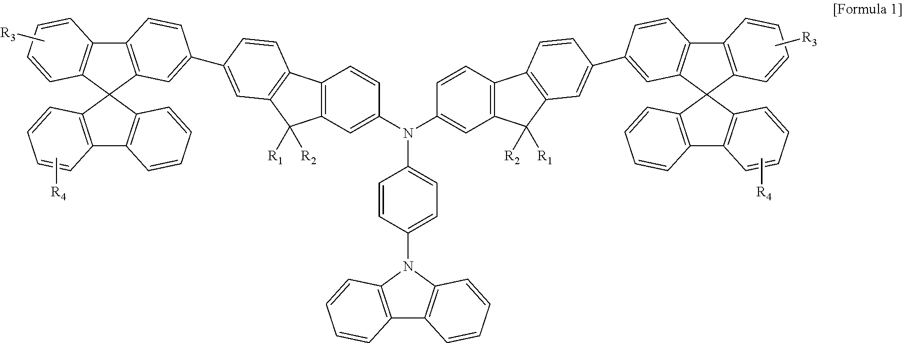

In the formula 1,

R₁ and R₂ are independently hydrogen or C₁˜C₂₀ alkyl group;

R₃ is hydrogen, C₁˜C₁₀ alkyl or cyano group; and

R₄ is hydrogen or C₁˜C₁₀ alkyl group.

Preferably, the hole transport layer composition for solar cell represented by formula 1 above can contain the compound of (1) or the compound of (2).

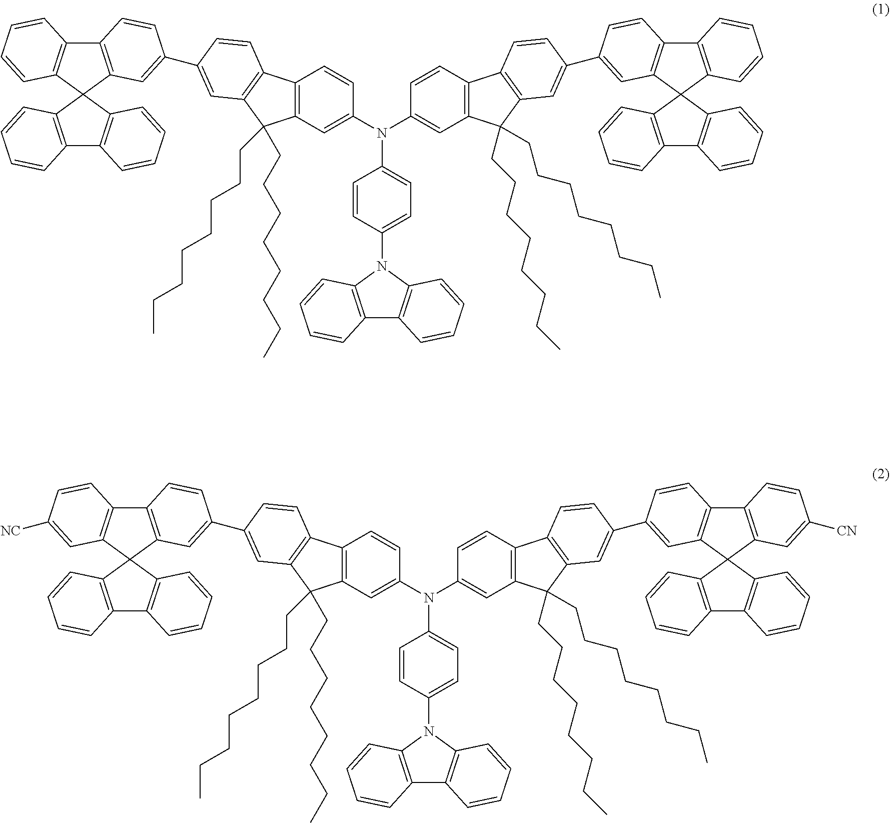 [SMILES: CCCCCCCCC1(CCCCCCCC)c2cc(-c3ccc4c(c3)C3(c5ccccc5-c5ccccc53)c3ccccc3-4)ccc2-c2ccc(N(c3ccc(-n4c5ccccc5c5ccccc54)cc3)c3ccc4c(c3)C(CCCCCCCC)(CCCCCCCC)c3cc(-c5ccc6c(c5)C5(c7ccccc7-c7ccccc75)c5ccccc5-6)ccc3-4)cc21 ; CCCCCCCCC1(CCCCCCCC)c2cc(-c3ccc4c(c3)C3(c5ccccc5-c5ccccc53)c3cc(C#N)ccc3-4)ccc2-c2ccc(N(c3ccc(-n4c5ccccc5c5ccccc54)cc3)c3ccc4c(c3)C(CCCCCCCC)(CCCCCCCC)c3cc(-c5ccc6c(c5)C5(c7ccccc7-c7ccccc75)c5cc(C#N)ccc5-6)ccc3-4)cc21]

The hole transport layer composition for solar cell represented by formula 1 of the present invention contains a compound having such a chemical structure that is formed with the main structure composed of phenylcarbazole and fluorene group connected each other by a nitrogen atom and with a spirobifluorene derivative as an end capper.

As an example, the hole transport layer composition of the invention can contain a compound having such a chemical structure that is formed with the main structure composed of phenylcarbazole and fluorene group connected each other by a nitrogen atom and having spirobifluorene (SBF) located at the end capper, which is 7-(9,9′-spirobi[fluorene]-2-yl)-N-(7-(9,9′-spirobi[fluorene]-2-yl)-9,9-dioctyl-9H-fluorene-2-yl)-N-(4-(9H-carbazole-9-yl)phenyl)-9,9-dioctyl-9H-fluorene-2-amine (CzPAF-SBF).

The hole transport layer composition of the invention can contain a compound having such a chemical structure that is formed with the main structure composed of phenylcarbazole and fluorene group connected each other by a nitrogen atom and having cyano group conjugated SBFN located at the end capper, which is 7-(7′-carbonitrile-9,9′-spirobi[fluorene]-2-yl)-N-(7-(7′-carbonitrile-9,9′-spirobi[fluorene]-2-yl)-9,9′-dioctyl-9H-fluorene-2-yl)-N-(4-9H-carbazole-9-yl)phenyl)-9,9-dioctyl-9H-fluorene-2-amine (CzPAF-SBFN).

The compound represented by formula 1 is characterized by a high hole mobility, a proper energy level, a thermo-stability, and an excellent solubility, and can be included as a hole transporting material in the Perovskite solar cell and bulk heterojunction organic solar cell.

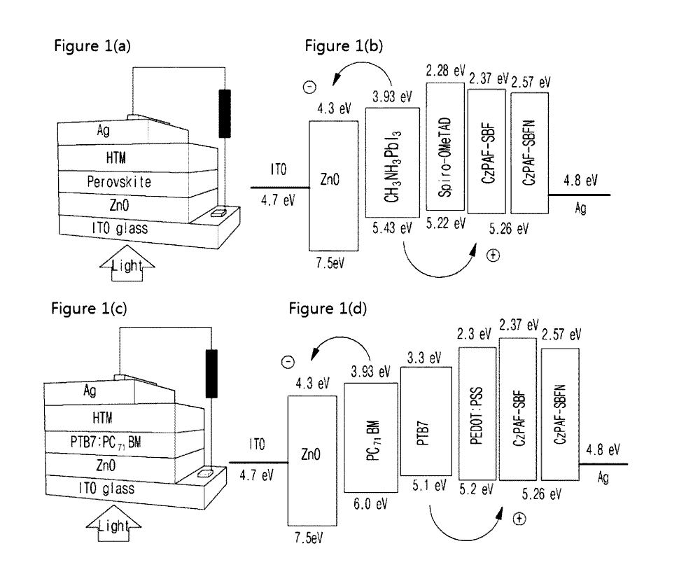

The open voltage of a solar cell is determined by the difference between the highest occupied molecular orbital (HOMO) of an electron donor and the lowest unoccupied molecular orbital (LUMO) of an electron acceptor. As shown in FIGS. 1(a)-1(d) below, HOMO and LUMO of CzPAF-SBF, a material that can be included in the hole transport layer composition of the invention, were measured to be −5.26 and −2.37 eV and HOMO and LUMO of CzPAF-SBFN were −5.27 and −2.57 eV.

The HOMO energy level of the compound of formula 1, for example CzPAF-SBF or CzPAF-SBFN, goes well with the energy level of CH₃NH₃PbI₃ (−5.43 eV) that can be included in the Perovskite layer of a solar cell, so that excellent charge separation and charge transfer in the interface between the hole transport layer and Perovskite layer can be expected.

This compound displays a similar HOMO energy level to that of the general hole transporting material spiro-OMeTAD (HOMO, −5.22 eV), due to the phenyl and fluorene rings introduced in the area of the nitrogen atom in HOMO.

That is, considering that open voltage depends on the difference between the HOMO level of a hole transporting material and the quasi-Fermi level of a metal oxide thin film, the hole transport layer composition of the present invention is expected to have higher open voltage than the conventional spiro-OMeTAD since it displays a similar HOMO level.

The present invention also provides a method for preparing a hole transport layer composition for solar cell comprising the following steps as shown in reaction formula 1:

preparing the compound represented by formula 3 by reacting carbazole represented by formula 2 with 4-iodoaniline (step 1);

preparing the compound represented by formula 5 by reacting the compound of formula 3 prepared in step 1) with the compound represented by formula 4 (step 2);

preparing the compound represented by formula 7 by reacting the compound of formula 5 prepared in step 2) with the compound represented by formula 6 (step 3); and

preparing the compound represented by formula 9 by reacting the compound of formula 7 prepared in step 3) with the compound represented by formula 8 (step 4);

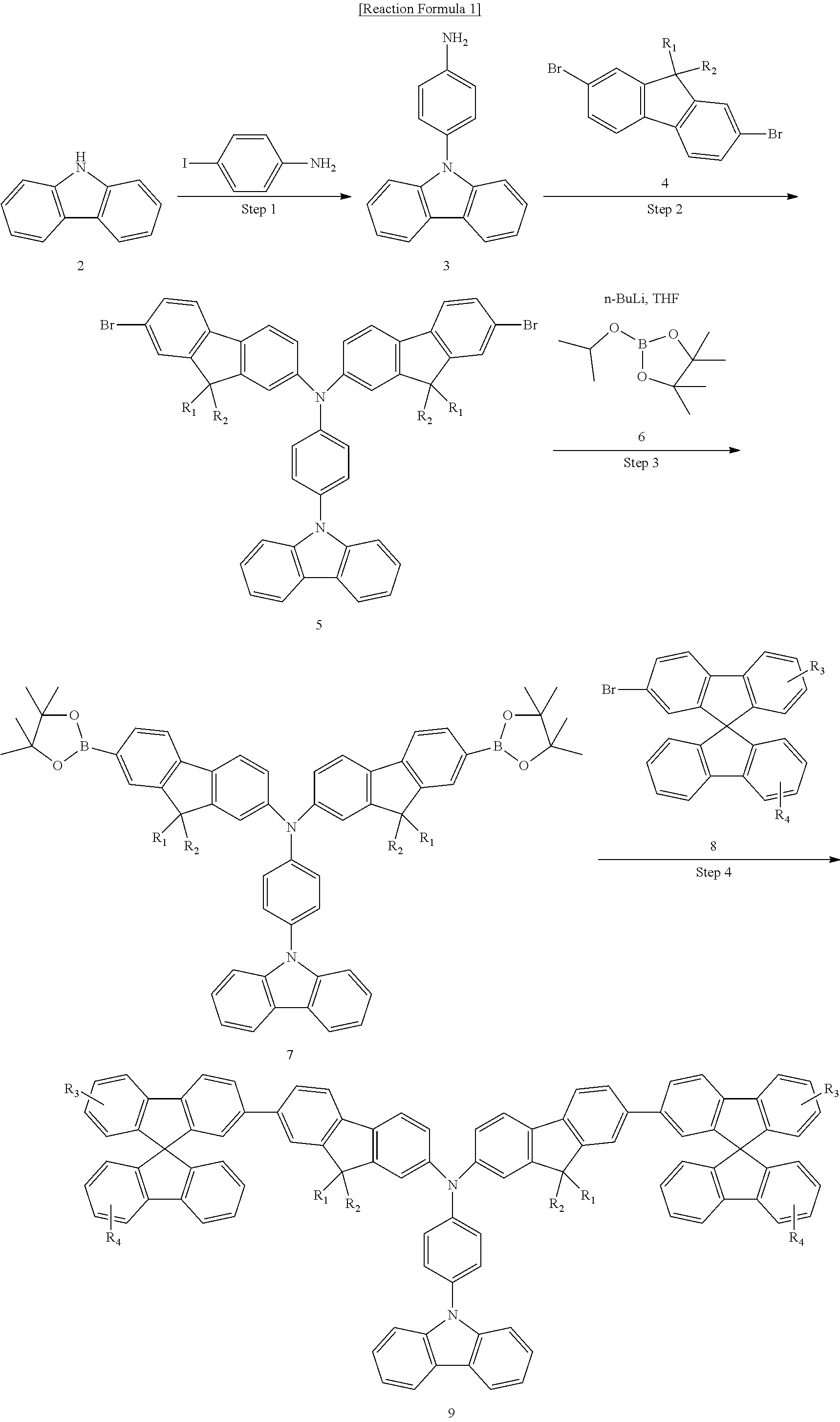

In the reaction formula 1,

R₁ and R₂ are independently hydrogen or C₁˜C₂₀ alkyl group;

R₃ is hydrogen, C₁˜C₁₀ alkyl or cyano group; and

R₄ is hydrogen or C₁˜C₁₀ alkyl group.

Hereinafter, the preparation method above is described in more detail, step by step.

In the reaction formula 1, step 1 is to give the compound represented by formula 3 by reacting carbazole represented by formula 2 with 4-iodoaniline.

At this time, copper oxide (Cu₂O) is used as a catalyst and diphenyl ether is used as a solvent, but not always limited thereto.

Further, the reaction in step 1) is induced at 150˜250° C. for 1˜24 hours, but not always limited thereto.

In the reaction formula 1 of the invention, step 2 is to give the compound represented by formula 5 by reacting the compound represented by formula 3 prepared in step 1) above with the compound represented by formula 4.

At this time, palladium acetate (Pd(OAc)₂) is used as a catalyst and toluene is used as an organic solvent, but not always limited thereto.

Further, the reaction in step 2) is induced at 150˜150° C. for 1˜24 hours, but not always limited thereto.

In the reaction formula 1 of the invention, step 3 is to give the compound represented by formula 7 by reacting the compound represented by formula 5 prepared in step 2) above with the compound represented by formula 6.

At this time, tetrahydrofuran (THF) is used as a solvent, but not always limited thereto.

Further, the reaction in step 3) is induced at −100˜0° C. for 1˜24 hours, but not always limited thereto. Herein, the room temperature indicates the general air temperature like 15˜25° C.

In the reaction formula 1 of the invention, step 4 is to give the compound represented by formula 9 by reacting the compound represented by formula 7 prepared in step 3) above with the compound represented by formula 8.

At this time, toluene is used as a solvent, but not always limited thereto.

Further, the reaction in step 4) is induced at 100˜150° C. for 1˜48 hours, but not always limited thereto.

The present invention also provides a solar cell comprising the compound represented by formula 1 as a hole transport layer material.

The present invention also provides a Perovskite solar cell comprising:

the first electrode contains a glass substrate;

the metal oxide layer formed on the first electrode above;

the Perovskite layer formed on the metal oxide layer above;

the hole transport layer formed on the Perovskite layer above; and

the second electrode formed on the hole transport layer above,

wherein the hole transport layer contains the hole transport layer composition.

The present invention also provides an organic solar cell comprising:

the first electrode contains a glass substrate;

the metal oxide layer formed on the first electrode above;

the photoactive layer formed on the metal oxide layer above;

the hole transport layer formed on the photoactive layer above; and

the second electrode formed on the hole transport layer above,

wherein the hole transport layer contains the hole transport layer composition.

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Preparation of a Hole Transport Layer Composition 1

As shown in reaction formula 2 below, 7-(9,9′-spirobi[fluorene]-2-yl)-N-(7-(9,9′-spirobi[fluorene]-2-yl)-9,9-dioctyl-9H-fluorene-2-yl)-N-(4-(9H-carbazole-9-yl)phenyl)-9,9-dioctyl-9H-fluorene-2-amine (CzPAF-SBF) was prepared. Particularly, CzPAF-SBF was prepared according to the following steps.

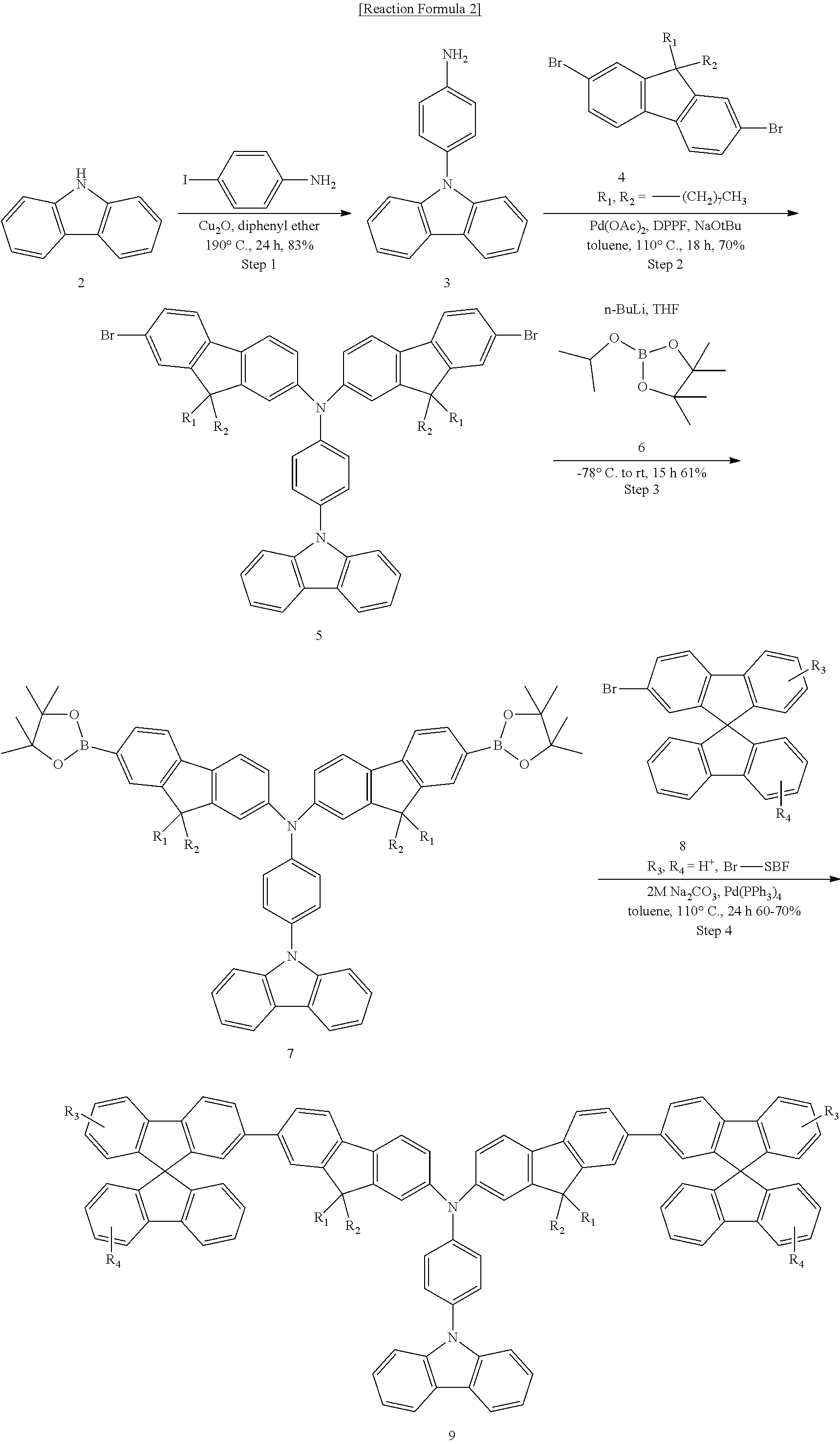

Step 1: Carbazole was reacted with 4-iodoaniline by using Cu₂O as a catalyst and diphenyl ether as a solvent at 190° C. for 24 hours to give the compound represented by formula 3 with the yield of 83%.

Step 2: Toluene (25 mL) containing the compound (0.60 g, 2.32 mmol) represented by formula 3 prepared in step 1), the compound (3.80 g, 6.97 mmol) represented by formula 4, and sodium tertiary butoxide (NaOtBu, 2.24 g, 23.25 mmol) was refluxed with nitrogen gas for 20 minutes, to which Pd(OAc)₂ (20.87 mg, 0.093 mmol) and 1,1′-bis(diphenylphosphino)ferrocene (DPPF) (103 mg, 0.186 mmol) were added thereafter. The reaction mixture was heated at 110° C. with stirring for 18 hours. Upon completion of the reaction, the mixture was diluted with diethyl ether (50 ml), which was filtered with celite bed. The filtrate was washed with diethyl ether twice. The filtered mixture was concentrated under reduced pressure, to which water (50 ml) was added, followed by extraction with diethyl ether (2×100 ml). The organic layer was washed with brine (50 ml) and dried over anhydrous sodium sulfate. The solvent was concentrated under reduced pressure. The residue was purified by column chromatography (silica gel; ethyl acetate/hexane=1/99) to give the compound 5 (CzPAF-Br, 1.90 g, 70%).

Step 3: n-BuLi (0.7 mL, 1.676 mmol, 2.5 M in hexane) was added to the dried THF (10 mL) containing the compound of formula 5 (CzPAF-Br, 500 mg, 0.419 mmol) prepared in step 2) at −78° C. The reaction mixture was stirred at that temperature for 45 minutes, to which 2-isopropoxy-4,4,5,6-tetramethyl-1,3,2-dioxaborolane (0.5 mL, 2.514 mmol) was quickly added. Then, the temperature of the mixture was raised to room temperature, followed by stirring for overnight. Upon completion of the reaction, the reaction mixture was slowly cooled down in cold water, followed by extraction with ethyl acetate (2×75 ml). The extract was washed with water and brine (50 mL). The organic layer was dried over sodium sulfate and the residue was purified by column chromatography (silica gel; ethyl acetate/hexane=2/98) to give the compound 7 (CzPAF-Borate, 330 mg, 61%).

Step 4: The mixture of the compound represented by formula 7 (CzPAF-Borate, 0.200 g, 0.155 mmol) prepared in step 3), the compound represented by formula 8 (Br-SBF, 0.246 g, 0.621 mmol), and Pd(PPh₃)₄ (9 mg, 0.007 mmol) was added to the solution comprising anhydrous toluene (20 mL) and Na₂CO₃ aqueous solution (15 mL, 2 M). The mixture was heated at 110° C., which was stirred for 24 hours in nitrogen atmosphere. Upon completion of the reaction, the reaction mixture was cooled down at room temperature. The organic layer was separated and the liquid phase was extracted with ethyl acetate. The mixed organic layer was washed with brine (2×75 ml) and then dried over anhydrous sodium sulfate. The solvent was concentrated under reduced pressure and the residue was purified by column chromatography (silica gel; ethyl acetate/hexane=10/90) to give the compound 9 (CzPAF-SBF, 0.180 g, 70%).

### Example 2

### Preparation of a Hole Transport Layer Composition 2

As shown in reaction formula 3 below, 7-(7′-carbonitrile-9,9′-spirobi[fluorene]-2-yl)-N-(7-(7′-carbonitrile-9,9′-spirobi[fluorene]-2-yl)-9,9′-dioctyl-9H-fluorene-2-yl)-N-(4-9H-carbazole-9-yl)phenyl)-9,9-dioctyl-9H-fluorene-2-amine (CzPAF-SBFN) was prepared. Particularly, CzPAF-SBFN was prepared according to the following steps.

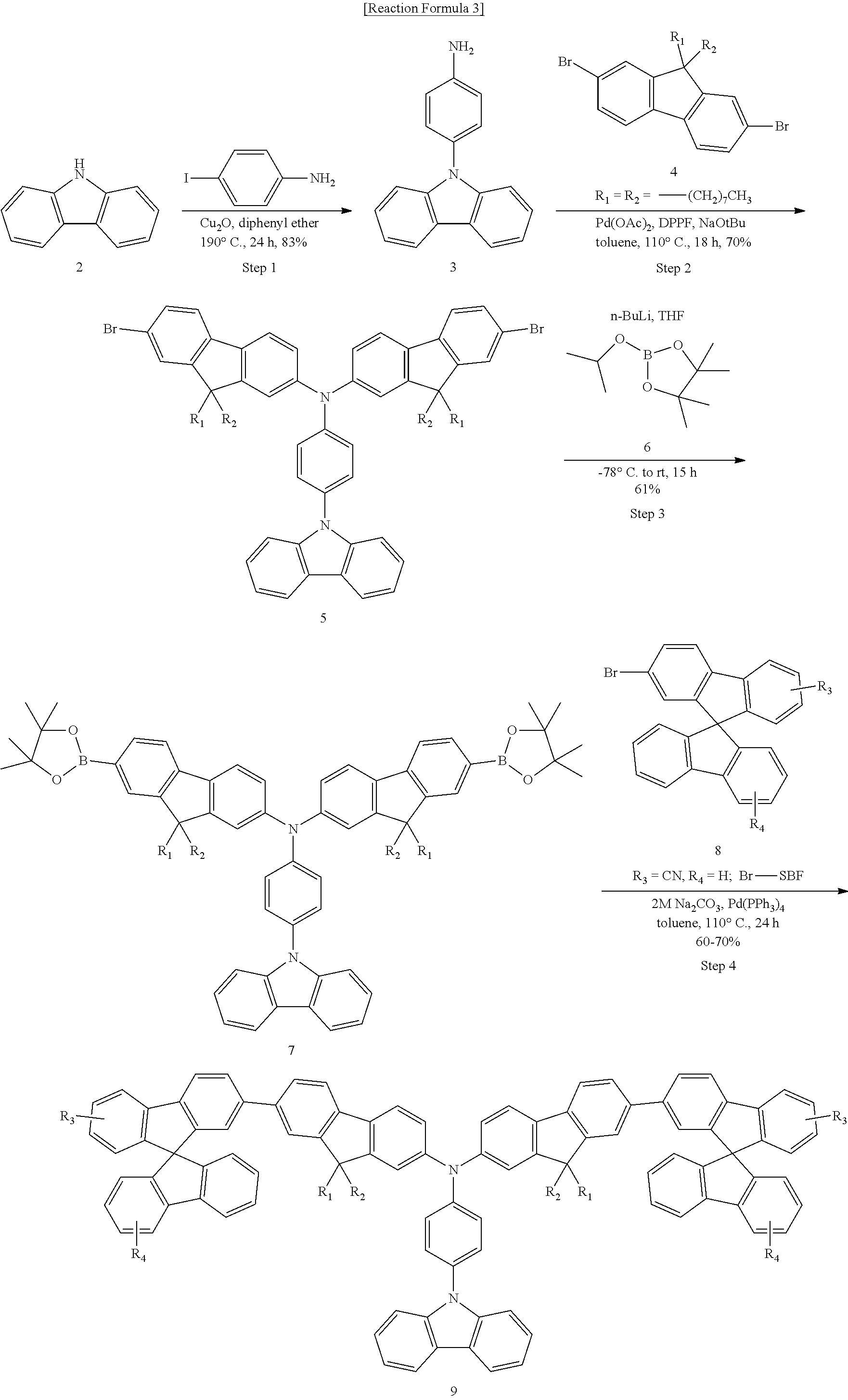

The compound represented by formula 9 was prepared by the same manner as described in Example 1 except step 4) of Example 1.

Step 4: The mixture of the compound represented by formula 7 (CzPAF-Borate, 0.150 g, 0.116 mmol) prepared in step 3) of Example 1, the compound represented by formula 8 (Br—SBFN, 0.195 g, 0.464 mmol), and Pd(PPh₃)₄ (6.7 mg, 0.005 mmol) was added to the solution comprising anhydrous toluene (15 mL) and Na₂CO₃ aqueous solution (10 mL, 2 M). The mixture was heated at 110° C., which was stirred for 24 hours in nitrogen atmosphere. Upon completion of the reaction, TLC was performed. The organic layer was separated and the liquid phase was extracted with ethyl acetate. The mixed organic layer was washed with brine (2×50 ml) and then dried over anhydrous sodium sulfate. The solvent was concentrated under reduced pressure and the residue was purified by column chromatography (silica gel; ethyl acetate/hexane=10/90) to give the compound 9 (CzPAF-SBFN, 0.120 g, 60%).

### Example 3

### Preparation of a Perovskite Solar Cell Comprising a Hole Transport Layer 1

A Perovskite solar cell comprising the compound of Example 1 as a hole transport layer was prepared. Particularly, a Perovskite solar cell was prepared according to the following steps.

Sep 1: An ITO substrate was coated with zinc oxide (ZnO) aqueous solution by spin coating at 3000 rpm for 30 seconds to form a zinc oxide layer in the thickness of 50 nm, which was heat-treated at 150° c. for 10 minutes.

Step 2: The zinc oxide layer was coated with 0.87 M PbI₂ solution (400 mg/mL in DMF) by spin coating at 6000 rpm for 30 seconds, which was dried on a 100° C. hot-plate.

Step 3: The layer coated with PbI₂ was coated with 40 mg of CH₃NH₃I dissolved in 1 mL of isopropyl alcohol (IPA) by spin coating at 6000 rpm for 30 seconds, which was dried on a 100° C. hot-plate for 1 minute.

Step 4: The film of step 3), MAPbI₃/ZnO/ITO film, was coated with the hole transporting material (CzPAF-SBF)/chlorobenzene solution prepared in Example 1 by spin coating at 4000 rpm for 30 seconds in the presence of the additives such as Li-TFS1 and t-BP to form a hole transport layer in the thickness of 200 nm.

Step 5: A silver (Ag) electrode was formed on the HTM/MAPbI₃/ZnO/ITO film of step 4) by using a thermal evaporator.

### Example 4

### Preparation of a Perovskite Solar Cell Comprising a Hole Transport Layer 2

A Perovskite solar cell was prepared by the same manner as described in Example 3 except that the CzPAF-SBFN prepared in Example 2 was used as a hole transporting material in step 4) of the method of Example 3.

### Example 5

### Preparation of an Organic Solar Cell Comprising a Hole Transport Layer 1

An organic solar cell comprising the compound of Example 1 as a hole transport layer was prepared. Particularly, an organic solar cell was prepared according to the following steps.

Sep 1: An ITO substrate was coated with zinc oxide (ZnO) aqueous solution by spin coating at 3000 rpm for 30 seconds to form a zinc oxide layer in the thickness of 50 nm, which was heat-treated at 150° C. for 10 minutes.

Step 2: Phenyl-C71-butyric acid methyl ester and Poly[4,8-bis[(2-ethylhexyl)oxy]benzo[1,2-b:4,5-b′]dithiophene-2,6-diyl][3-fluoro-2-[(2-ethylhexyl)carbonyl]thieno[3,4-b]thiophenediyl] (PTB7) were mixed at the ratio of 12 mg:8 mg in 0.97 mL of chlorobenzene (CB). 0.03 mL of 1,8-diiodooctane (DIO) solution was added thereto. The mixture was stirred at 60° C. for 12 hours. A photoactive layer in the thickness of 100 nm was formed on the ZnO conductive film.

Step 3: The hole transporting material (CzPAF-SBF) prepared in Example 1 was diluted in PIA (1 mg:10 mL) and a thin P-type conducting film was formed on the photoactive layer above.

Step 4: A silver (Ag) electrode in the thickness of 120 nm was formed on the HTM/photoactive layer/ZnO/ITO film of step 4) by using a thermal evaporator.

### Example 6

### Preparation of an Organic Solar Cell Comprising a Hole Transport Layer 2

An organic solar cell was prepared by the same manner as described in Example 5 except that the CzPAF-SBFN prepared in Example 2 was used as a hole transporting material in step 3) of the method of Example 5.

### Comparative Example 1

Spiro-OMeTAD, a material usable as a hole transport layer composition for the conventional solar cell and having the structure shown in formula 10, was prepared.

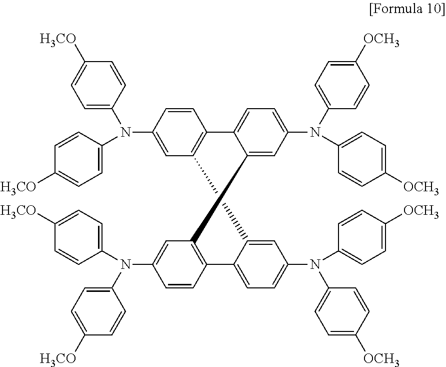

### Comparative Example 2

A Perovskite solar cell was prepared by the same manner as described in Example 3 except that the Spiro-OMeTAD of Comparative Example 1 was used as a hole transporting material in step 4) of the method of Example 3.

### Comparative Example 3

An organic solar cell was prepared by the same manner as described in Example 5 except that the Spiro-OMeTAD of Comparative Example 1 was used as a hole transporting material in step 3) of the method of Example 5.

### Experimental Example 1

### UV-VIS Absorption and Fluorescence Spectrum

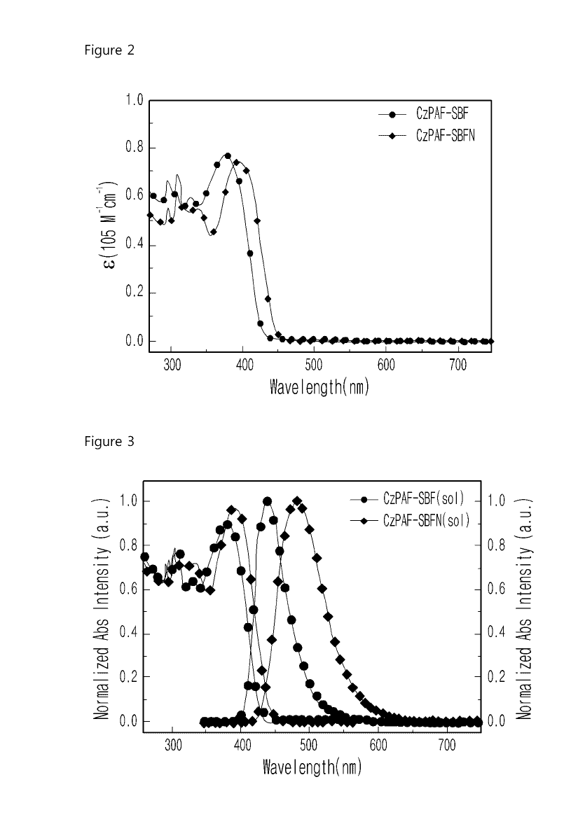

(1) To investigate the UV-VIS absorption spectrum and the electrical characteristics of the hole transporting materials CzPAF-SBF and CzPAF-SBFN prepared in Examples 1 and 2, these hole transporting materials were analyzed with an absorption spectrometer (JASCO, V-570) in chloroform aqueous solution (1×10⁻⁵ M). The results are shown in FIG. 2.

As shown in FIG. 2, the maximum absorption (λₘₐₓ^{a}) was shown at 377 nm (molar absorptivity ε=77049 L mol⁻¹cm⁻¹) in the absorption-emission spectrum of CzPAF-SBF in chloroform aqueous solution and the maximum absorption (λₘₐₓ^{a}) was observed at 396 nm (molar absorptivity ε=74147 L mol⁻¹cm⁻¹) in the absorption-emission spectrum of CzPAF-SBFN in chloroform aqueous solution. The maximum emission (λₘₐₓ^{b}) was shown at 441 nm in the fluorescence spectrum of CzPAF-SBF in chloroform aqueous solution and the maximum emission (λₘₐₓ^{b}) was observed at 487 nm in the fluorescence spectrum of CzPAF-SBFN in chloroform aqueous solution.

Further, the hole mobility of CzPAF-SBF was 3.09×10⁻⁴ cm²V⁻¹s⁻¹ and the hole mobility of CzPAF-SBFN was 1.28×10⁻⁴ cm²V⁻¹s⁻¹.

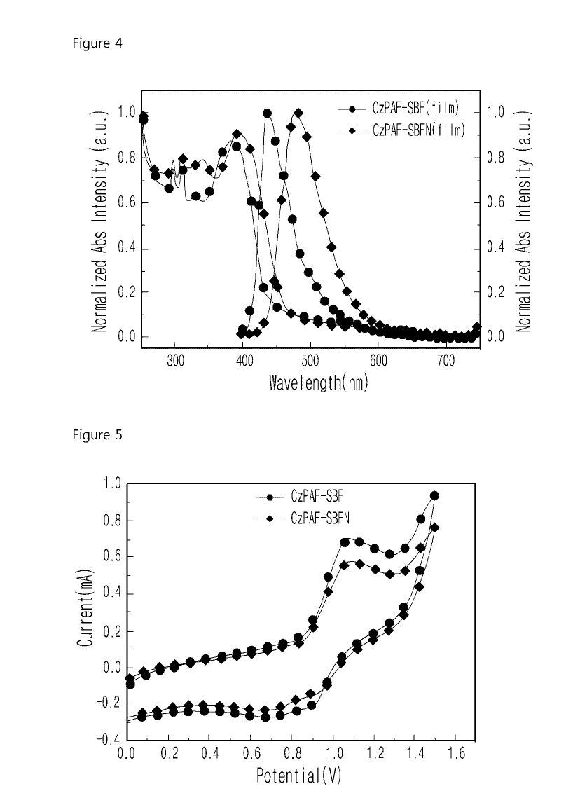

(2) The UV-VIS absorption spectrum and the fluorescence spectrum of the hole transporting materials CzPAF-SBF and CzPAF-SBFN prepared in Examples 1 and 2 were recorded by using an absorption spectrometer (JASCO, V-570) in chloroform aqueous solution and as a film. The results are shown in the graphs of FIGS. 3 and 4.

As shown in FIGS. 3 and 4, the absorption spectrums of CzPAF-SBF and CzPAF-SBFN, as a film, were similar to those observed as in chloroform aqueous solution, suggesting that the hole transporting materials developed in the present invention, CzPAF-SBF and CzPAF-SBFN, have a weak inter-molecular interaction.

### Experimental Example 2

### Cyclic Voltametry

To investigate the HOMO and LUMO energy levels of the hole transporting materials prepared in Examples 1 and 2, CzPAF-SBF and CzPAF-SBFN, cyclic voltametry was performed and the results are shown in FIG. 5.

As shown in FIG. 5, the two compounds demonstrated irreversible oxidation and both compounds had their oxidation peak at 1.07 V.

In the meantime, the photonic band gaps (Eg^{opt c}) of the hole transporting materials prepared in Examples 1 and 2, CzPAF-SBF and CzPAF-SBFN, were respectively 2.89 and 2.76 eV. HOMO and LUMO of CzPAF-SBF were −5.26 and −2.37 eV. HOMO and LUMO of CzPAF-SBFN were −5.27 and −2.57 eV. Unlike the similar HOMO levels between the two compounds, the LUMO levels were different, which seemed because the cyano group introduced in SBFN of CzPAF-SBFN caused electron-withdrawing so as to strongly affect the distribution of LUMO levels.

The optical and electrical characteristics of CzPAF-SBF and CzPAF-SBFN shown in Experimental Examples 1 and 2 were summarized in Table 1 below.

TABLE 1

| | | | | | | | Hole |
|---|---|---|---|---|---|---|---|
| | λₘₐₓ^{a} | ε | λₘₐₓ^{b} | Eg^{optc} | HOMO^{d} | LUMO^{d} | mobility |
| | (nm) | (M⁻¹cm⁻¹) | (nm) | (eV) | (eV) | (eV) | (cm²V⁻¹s⁻¹) |
| CzPAF-SBF | 377 | 77049 | 441 | 2.89 | −5.26 | −2.37 | 3.09 × 10⁻⁴ |
| CzPAF-SBFN | 396 | 74147 | 487 | 2.76 | −5.27 | −2.57 | 1.28 × 10⁻⁴ |

### Experimental Example 3

### Thermogravimetric Analysis and Differential Scanning Calorimetry

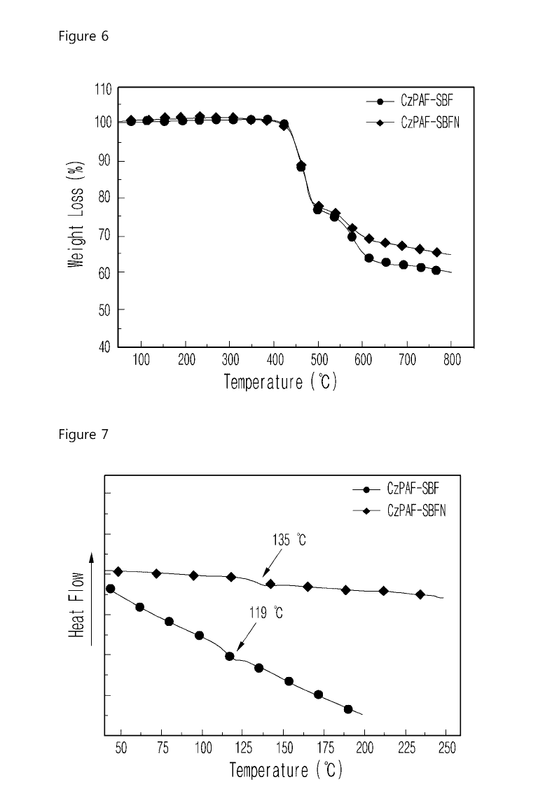

To investigate the thermal characteristics of the hole transporting materials prepared in Examples 1 and 2, CzPAF-SBF and CzPAF-SBFN, thermogravimetric analysis (TGA, Mettler Toledo, TGA/SDTA) and differential scanning calorimetry (DSC, Mettler Toledo, DSC 822e) were performed. The results are shown in the graphs of FIGS. 6 and 7.

As shown in FIGS. 6 and 7, the decomposition temperatures (Td) of the two compounds where the degradation started and progressed about 5% by the weights of CzPAF-SBF and CzPAF-SBFN were 446 and 447° C. Such a high decomposition temperature indicates that the two compounds have a thermo-stability.

The glass transition temperatures of CzPAF-SBF and CzPAF-SBFN were 119° C. and 135° C. From the above results, it was confirmed that the composition of the present invention is useful for the formation of a non-crystalline film which has higher efficiency and stability particularly thermo-stability since the composition contains a compound comprising the SBF structure.

### Experimental Example 4

### Nuclear Magnetic Resonance Spectroscopy

(1) The compound represented by formula 5 in reaction formula 2 of Example 1 was analyzed by nuclear magnetic resonance spectroscopy (Varian Mercury, Plus 300) and the results are presented below.

¹H NMR (300 MHz, CDCl₃, δ): 8.149 (d, J=7.8 Hz, 2H), 7.584 (d, J=8.1 Hz, 3H), 7.506-7.409 (m, 11H), 7.327-7.234 (m, 6H), 7.13 (d, J=7.5 Hz, 2H), 1.906-1.858 (m, 8H), 1.133-1.032 (m, 40H), 0.788 (t, J=6.6 Hz, 12H), 0.693 (m, 8H).

(2) The compound represented by formula 7 in reaction formula 2 of Example 1 was analyzed by nuclear magnetic resonance spectroscopy (Varian Mercury, Plus 300) and the results are presented below.

¹H NMR (300 MHz, CDCl₃, δ): 8.151 (d, J=7.8 Hz, 2H), 7.812 (d, J=7.8 Hz, 2H), 7.718-7.630 (m, 5H), 7.502-7.415 (m, 6H), 7.361-7.246 (m, 7H), 7.15 (d, J=7.5 Hz, 2H), 1.906-1.857 (m, 8H), 1.39 (s, 24H), 1.133-1.032 (m, 40H), 0.773 (t, J=6.0 Hz, 12H), 0.657 (m, 8H).

(3) The compound represented by formula 9 in reaction formula 2 of Example 1 was analyzed by nuclear magnetic resonance spectroscopy (Varian Mercury, Plus 300) and the results are presented below.

¹H NMR (300 MHz, CDCl₃, δ): 8.165-8.139 (d, J=7.8 Hz, 2H), 7.952-7.877 (m, 8H), 7.709-7.684 (d, J=7.5 Hz, 2H), 7.582-7.469 (m, 7H), 7.453-7.363 (m, 14H), 7.338-7.289 (m, 4H), 7.175-7.091 (m, 9H), 7.048 (s, 2H), 6.841-6.816 (d, J=7.5 Hz, 4H), 6.735-6.710 (d, J=7.5 Hz, 2H), 1.901-1.878 (m, 8H), 1.133-1.031 (m, 40H), 0.757 (t, J=6.0 Hz, 12H), 0.673 (m, 8H);

¹³C NMR (75 MHz, CDCl₃, 5): 152.538, 151.175, 149.291, 148.816, 147.376, 146.595, 141.831, 141.433, 141.341, 140.942, 140.008, 139.181, 136.393, 131.260, 128.382, 128.167, 127.677, 127.125, 125.854, 124.368, 124.031, 123.801, 123.250, 122.652, 122.315, 121.182, 120.477, 120.171, 119.834, 119.206, 109.800, 66.113, 55.191, 40.256, 31.739, 30.008, 29.318, 29.211, 23.942, 22.578, 14.092.

(4) The compound represented by formula 9 in reaction formula 3 of Example 2 was analyzed by nuclear magnetic resonance spectroscopy (Varian Mercury, Plus 300) and the results are presented below.

¹H NMR (300 MHz, CDCl₃, δ): 8.157-8.133 (d, J=7.2 Hz, 2H), 7.984-7.894 (m, 8H), 7.747-7.658 (m, 5H), 7.579-7.528 (m, 5H), 7.443-7.307 (m, 16H), 7.282-7.076 (m, 10H), 6.972 (m, 2H), 6.792-6.767 (d, J=7.5 Hz, 4H), 1.901-1.878 (m, 8H), 1.133-1.031 (m, 40H), 0.746 (m, 20H);

¹³C NMR (75 MHz, CDCl₃, 5): 152.523, 151.298, 150.164, 149.980, 147.116, 146.748, 145.768, 143.363, 141.862, 140.881, 140.498, 138.859, 138.553, 136.087, 132.165, 131.261, 128.382, 128.167, 127.600, 126.283, 125.839, 124.031, 123.587, 123.219, 122.698, 121.366, 121.151, 120.676, 120.539, 120.431, 119.788, 119.313, 119.175, 110.551, 109.755, 66.067, 55.222, 40.240, 31.769, 30.008, 29.349, 29.242, 23.926, 22.609, 14.092.

### Experimental Example 5

### Mass Spectrometry

(1) The compound represented by formula 9 in reaction formula 2 of Example 1 was analyzed by mass spectrometry (FAB Mass, Korea Basic Science and Institute Daejeon Center) and the results are presented below.

MS (FAB): m/z (100%): calcd for C₁₂₆H₁₂₂N₂, 1163.96. found, 1663.97. Anal. calcd for C₁₂₆H₁₂₂N₂: C, 90.93; H, 7.39; N, 1.68. found: C, 90.78; H, 7.31; N, 1.77.

(2) The compound represented by formula 9 in reaction formula 3 of Example 2 was analyzed by mass spectrometry (FAB Mass, Korea Basic Science and Institute Daejeon Center) and the results are presented below.

MS (FAB): m/z (100%): calcd for C₁₂₈H₁₂₀N₄ 1713.95. found, 1713.95. Anal. calcd for C₁₂₈H₁₂₀N₄: C, 89.68; H, 7.06; N, 3.27. found: C, 89.57; H, 7.00; N, 3.47.

### Experimental Example 6

### Characteristics of Perovskite Solar Cell According to the Kind of a Hole Transporting Material

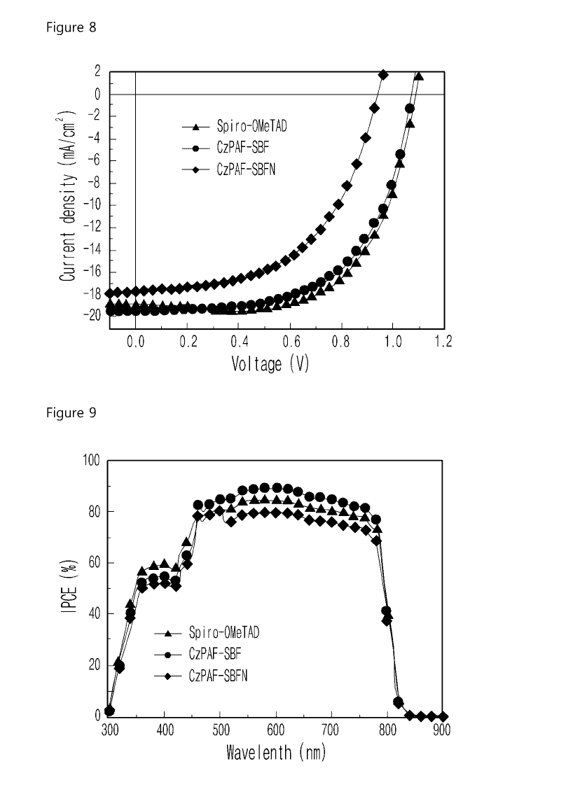

The current efficiency (IPCE) and the photocurrent density-voltage (J-V) curve of the Perovskite solar cells of Examples 3 and 4 and Comparative Example 2 were analyzed by solar simulator. The results are shown in FIGS. 8 and 9. Also, the power conversion efficiency (PCE) of the Perovskite solar cells of Examples 3 and 4 and Comparative Example 2 was calculated from open voltage, short-circuit current, and charging rate. The results are shown in Table 2.

As shown in FIGS. 8 and 9, from the results of the analysis of the IPCE of the Perovskite solar cells of Examples 3 and 4 and Comparative Example 2, it was confirmed that the IPCE of the solar cell of Example 3 was approximately 85%, which was higher than that of the solar cell of Comparative Example 2.

As shown in Table 2, the power conversion efficiency (PCE) of the Perovskite solar cells of Examples 3 and 4 and Comparative Example 2 was measured and the average of the measured values obtained from 9 times repeated measurement was calculated. At this time, the power conversion efficiency of the solar cell of Example 3 was approximately 15.16%, which was as much improved as 3.6% from the value of the solar cell of Comparative Example 2.

TABLE 2

| | V_{oc} | J_{sc} | FF | Rₛ | PCE |
|---|---|---|---|---|---|
| HTM | (V) | (mAcm⁻²) | (%) | (Ωcm²) | (%) |
| Spiro- | 1.05 ± 0.03 | 19.43 ± 2.35 | 64.21 ± 2.59 | 3.21 ± 1.05 | 13.00 ± 1.51 |
| OMeTAD | | | | | (14.61) |
| CzPAF-SBF | 1.05 ± 0.03 | 19.69 ± 2.69 | 65.07 ± 3.05 | 2.87 ± 0.87 | 13.55 ± 1.61 |
| | | | | | (15.16) |
| CzPAF-SBFN | 1.04 ± 0.03 | 18.51 ± 1.51 | 59.87 ± 2.57 | 5.32 ± 1.92 | 11.55 ± 0.94 |
| | | | | | (12.49) |

### Experimental Example 7

### Characteristics of Bulk Heterojunction Organic Solar Cell According to the Kind of a Hole Transporting Material

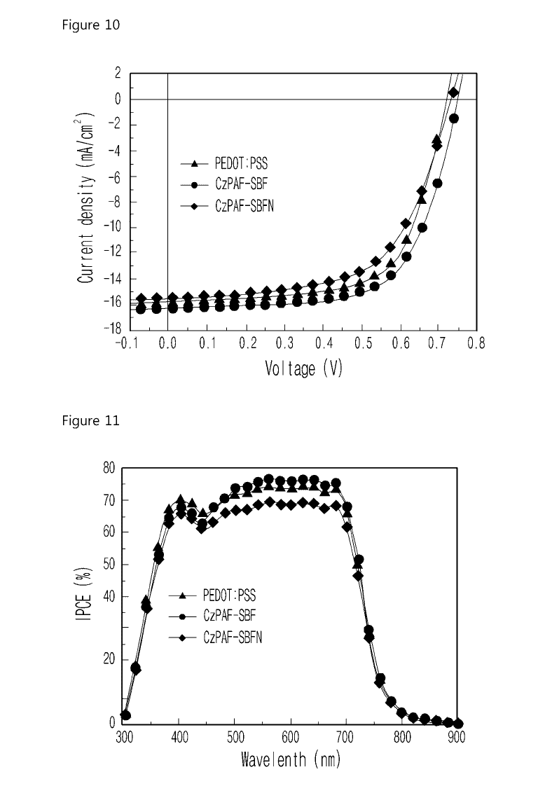

The current efficiency (IPCE) and the photocurrent density-voltage (J-V) curve of the bulk heterojunction organic solar cells of Examples 5 and 6 and Comparative Example 3 were analyzed by solar simulator. The results are shown in FIGS. 10 and 11. Also, the power conversion efficiency (PCE) of the bulk heterojunction organic solar cells of Examples 5 and 6 and Comparative Example 3 was calculated from open voltage, short-circuit current, and charging rate. The results are shown in Table 3.

As shown in FIGS. 10 and 11, from the results of the analysis of the IPCE of the bulk heterojunction organic solar cells of Examples 5 and 6 and Comparative Example 3, it was confirmed that the IPCE of the solar cell of Example 5 was approximately 80%, which was higher than that of the solar cell of Comparative Example 3.

As shown in Table 3, the power conversion efficiency (PCE) of the bulk heterojunction organic solar cells of Examples 5 and 6 and Comparative Example 3 was measured and the average of the measured values obtained from 9 times repeated measurement was calculated. At this time, the power conversion efficiency of the solar cell of Example 5 was approximately 7.93%, which was as much improved as 2.3% from the value of the solar cell of Comparative Example 3.

TABLE 3

| | V_{oc} | J_{sc} | FF | PCE |
|---|---|---|---|---|
| HTM | (V) | (mAcm⁻²) | (%) | (%) |
| PEDOT:PSS | 0.73 ± 0.01 | 16.26 ± 0.30 | 64.28 ± 1.51 | 7.63 ± 0.12 |
| | | | | (7.74) |
| CzPAF-SBF | 0.74 ± 0.01 | 16.31 ± 0.22 | 64.86 ± 1.35 | 7.85 ± 0.08 |
| | | | | (7.93) |
| CzPAF- | 0.73 ± 0.01 | 15.43 ± 0.12 | 57.74 ± 2.25 | 6.49 ± 0.30 |
| SBFN | | | | (6.80) |

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended Claims.

What is claimed is:

1. A hole transport layer composition for solar cell comprising the compound represented by formula 1 below. 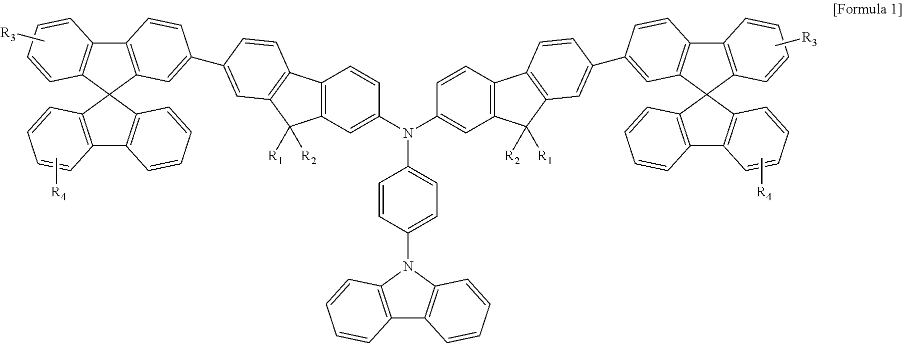
(In the formula 1,
R₁ and R₂ are independently hydrogen or C₁˜C₂₀ alkyl group;
R₃ is hydrogen, C₁˜C₁₀ alkyl or cyano group; and
R₄ is hydrogen or C₁˜C₁₀ alkyl group.)

2. The hole transport layer composition for solar cell according to claim 1, wherein the hole transport layer composition for solar cell represented by formula 1 is the compound of (1) or the compound of (2).

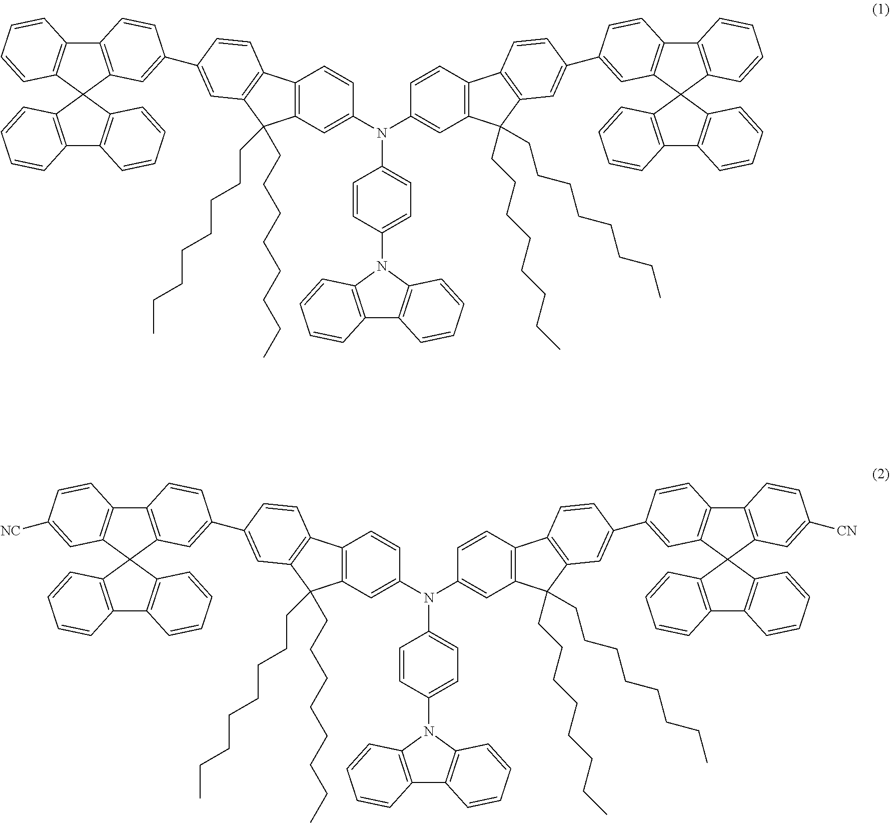 [SMILES: CCCCCCCCC1(CCCCCCCC)c2cc(-c3ccc4c(c3)C3(c5ccccc5-c5ccccc53)c3ccccc3-4)ccc2-c2ccc(N(c3ccc(-n4c5ccccc5c5ccccc54)cc3)c3ccc4c(c3)C(CCCCCCCC)(CCCCCCCC)c3cc(-c5ccc6c(c5)C5(c7ccccc7-c7ccccc75)c5ccccc5-6)ccc3-4)cc21 ; CCCCCCCCC1(CCCCCCCC)c2cc(-c3ccc4c(c3)C3(c5ccccc5-c5ccccc53)c3cc(C#N)ccc3-4)ccc2-c2ccc(N(c3ccc(-n4c5ccccc5c5ccccc54)cc3)c3ccc4c(c3)C(CCCCCCCC)(CCCCCCCC)c3cc(-c5ccc6c(c5)C5(c7ccccc7-c7ccccc75)c5cc(C#N)ccc5-6)ccc3-4)cc21]

3. A method for preparing the hole transport layer composition for solar cell of claim 1 comprising the following steps as shown in reaction formula 1:
preparing the compound represented by formula 3 by reacting carbazole represented by formula 2 with 4-iodoaniline (step 1);
preparing the compound represented by formula 5 by reacting the compound of formula 3 prepared in step 1) with the compound represented by formula 4 (step 2);
preparing the compound represented by formula 7 by reacting the compound of formula 5 prepared in step 2) with the compound represented by formula 6 (step 3); and
preparing the compound represented by formula 9 by reacting the compound of formula 7 prepared in step 3) with the compound represented by formula 8 (step 4); 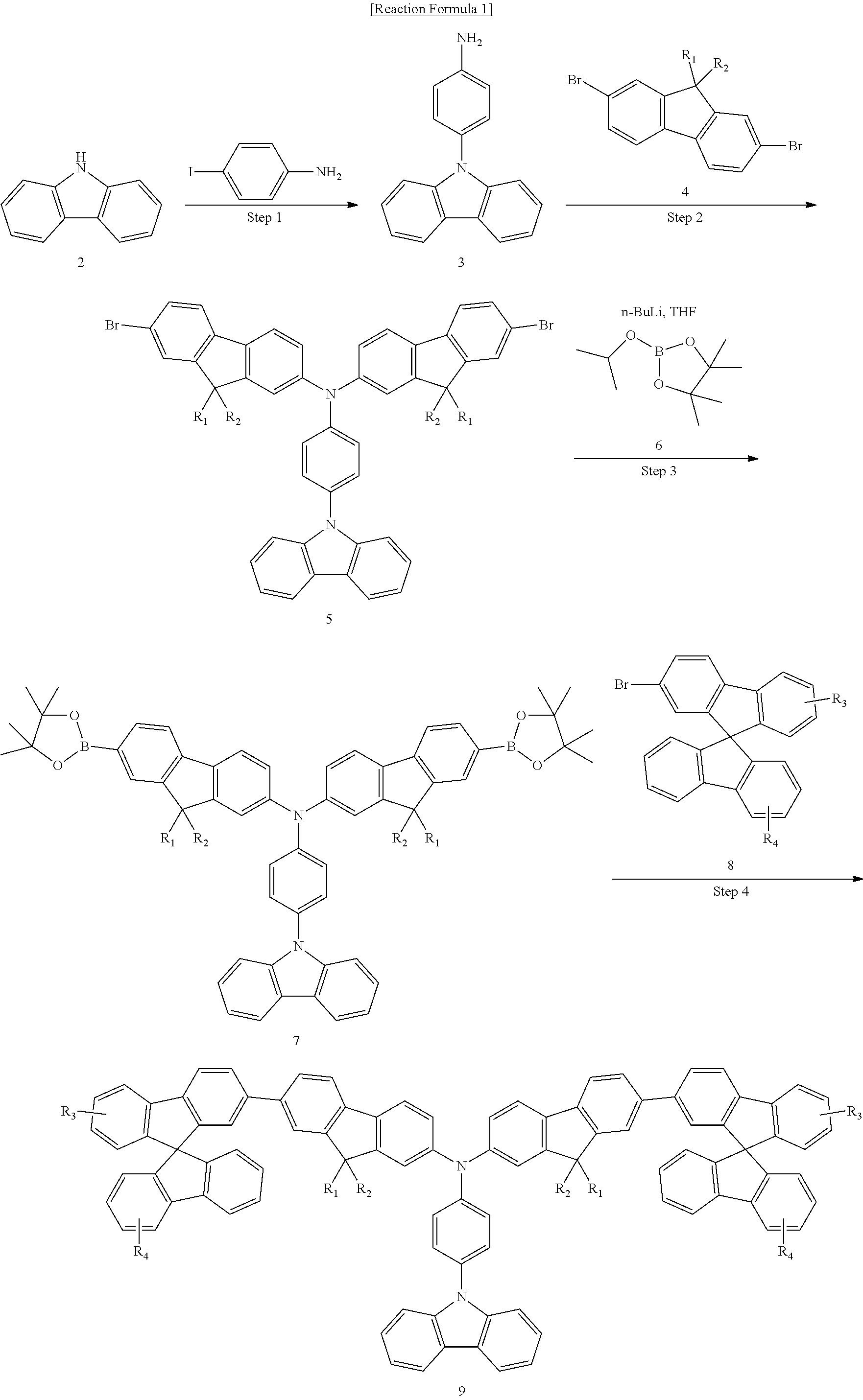
(In the reaction formula 1,
R₁ and R₂ are independently hydrogen or C₁˜C₂₀ alkyl group;
R₃ is hydrogen, C₁˜C₁₀ alkyl or cyano group; and
R₄ is hydrogen or C₁˜C₁₀ alkyl group.)

4. A Perovskite solar cell comprising:
the first electrode contains a glass substrate;
the metal oxide layer formed on the first electrode above;
the Perovskite layer formed on the metal oxide layer above;
the hole transport layer formed on the Perovskite layer above; and
the second electrode formed on the hole transport layer above,
wherein the hole transport layer contains the hole transport layer composition of claim 1.

5. An organic solar cell comprising:
the first electrode contains a glass substrate;
the metal oxide layer formed on the first electrode above;
the photoactive layer formed on the metal oxide layer above;
the hole transport layer formed on the photoactive layer above; and
the second electrode formed on the hole transport layer above,
wherein the hole transport layer contains the hole transport layer composition of claim 1.
